(19) United States
(12) Patent Application Publication
(10) Pub. No.: US 2013/0116338 A1
(43) Pub. Date: May 9, 2013

(54) NANOMETAL DISPERSION AND METHOD FOR PREPARING THE SAME

(71) Applicants: Chih-Wei CHOU, Taichung City (TW); Ya-Hsun HOU, Shuishang Township (TW); Yueh-Hsiung KUO, Taipei City (TW); Yung-Hsiu CHEN, Kaohsiung City (TW)

(72) Inventors: Chih-Wei CHOU, Taichung City (TW); Ya-Hsun HOU, Shuishang Township (TW); Yueh-Hsiung KUO, Taipei City (TW); Yung-Hsiu CHEN, Kaohsiung City (TW)

(73) Assignee: CHINA MEDICAL UNIVERSITY, Taichung City (TW)

(21) Appl. No.: 13/289,439

(22) Filed: Nov. 4, 2011

(51) Int. Cl. A61K 47/00; B82Y 5/00

(52) U.S. Cl. 514/769; 977/700; 977/906

(57) ABSTRACT

A nanometal dispersion and a method for preparing a nanometal dispersion are provided. The method comprises the following steps: providing a first solution containing a metal ion; providing a second solution containing inulin; mixing the first solution and the second solution to provide a third solution; and providing energy to the third solution to conduct a reduction-oxidation reaction to form a nanometal therein. The produced nanometal dispersion comprises inulin and a nanometal with multimorphology.

### CROSS-REFERENCES TO RELATED APPLICATIONS

Not applicable.

### BACKGROUND OF THE INVENTION

1. Field of the Invention

The present invention relates to a nanometal dispersion and a method for preparing the dispersion. In particular, the present invention relates to a dispersion with good dispersibility and high biocompatibility, wherein the dispersion comprises inulin and a multimorphological nanometal.

2. Descriptions of the Related Art

It is well known that when the size of a bulk material is reduced down to a nano scale, its original physical properties such as optical, electrical, magnetic, and mechanical properties would change dramatically. For example, the melting point of pure gold has a fixed value (about 1064° C.) but as the particle size is reduced down to nano scale, there is no longer a fixed value. See Ph. Buffat et al., Size Effect of the Melting Temperature of Gold Particles, Phys. Rev. A, 1976, 13, 2287, which is incorporated hereinto by reference. According to the previous facts, it opens a field for nano material applications.

Nanometals have applications in many fields. For example, in many important catalysts that are constituted by metals, nano-scaled metals would greatly enhance the performance of the catalysts. For example, it is hard to apply a bulk of gold into a chemical reaction, but gold can be used as an excellent catalyst for the oxidation of carbon monoxide at low temperatures when its particle size is about 2 nm. In addition, the surface plasma resonance of a nanometal demonstrates its unique and strong light absorption character, and such character is affected by molecules adsorbed on the surface of the nanometal, and thus, the nanometal can be used as a sensor. In medical engineering, nanometals can be used for the diagnosis, treatment, and prevention of diseases and in areas like drug delivery, medical detection, gene detection, etc. For example, molecules, like DNA or proteins, are attached onto nanometal particles and through the alternation of fluorescence, conductivity and magnetism, it can be used for the diagnosis and assistance of treatment. The high specific surface area of nanometal particles can enhance the sensitivity for detection of diseases in the early stages and therefore, only damage cancer cells selectively. Among the variety of metals, gold has a high biocompatibility and is particularly being used in biolabeling and detection.

It is known that the property and application of a nanometal can change in accordance with its morphology. For example, in medical engineering, it has been reported that as compared with gold nanospheres, gold nanoparticles with hexagon and boot shapes exhibit high-sensitivity surface-enhanced Raman scattering (SERS) and have been successfully applied to the detection of Avidin (an egg white protein). See Jianqiang Hu et al., Gold Nanoparticles with Special Shapes: Controlled Synthesis, Surface-enhanced Raman Scattering, and The Application in Biodetection, Sensors, 2007, 7, 3299-3311, which is incorporated hereinto by reference.

Currently, many methods for preparing a nanometal have been proposed, including the laser ablation method, metal vapor synthesis, chemical reduction method, etc. In the laser ablation method, the high energy of the laser is used to melt metal, and through the low temperature environment provided by the solution and stabilizing agent contained therein, the nanometal formed can be evenly dispersed in the solution. In metal vapor synthesis, the main principle is to atomize a metal into metal atom steam and then mix the metal atom steam with an inert gas or an organic steam. The metal atom steam is then condensed onto a clean surface at a low temperature, followed by a separation procedure to obtain a nanometal. In the chemical reduction method, the oxidized metal ion is reduced back into a zero-charged metal by a reducing agent or an electrochemical system and the growth of the desired nanometal can be controlled with relevant operation conditions.

The chemical reduction method includes the commonly-used seed-mediated growth method. The principle of the seed-mediated growth method is based on using small-sized nanometals (usually in the range from a few nanometers to tens of nanometers) as a seed crystal and adding a reducing agent to allow the metal ion to be reduced and then to grow to the desired size and morphological nanometal on the seed crystal. Anand Gole et al. have disclosed a method for preparing nanometals via seed-mediated synthesis. (See Seed-Mediated Synthesis of Gold Nanorods: Role of the Size and nature of the Seed, Chem. Mater., 2004, 16, 3633-3640, which is incorporated hereinto by reference.) It is necessary for such a method to additionally formulate a growth solution for synthesizing gold nanorods with a step by step reaction. However, this method is extremely time-consuming, costly and extremely complicated in procedure. Also, only a single-morphological nanometal would be obtained, and therefore, it is hard to promote such a method due to its low applicability.

Based on the above needs, this invention provides a simple preparation method that could obtain a multimorphological nanometal-containing dispersion with good dispersibility on a larger scale.

### SUMMARY OF THE INVENTION

One objective of this invention is to provide a method for preparing a nanometal dispersion, comprising the following steps: providing a first solution containing a metal ion; providing a second solution containing inulin; mixing the first solution and the second solution to provide a third solution; and providing energy to the third solution to conduct a reduction-oxidation reaction to form a nanometal therein.

Another objective of this invention is to provide a nanometal dispersion comprising inulin and a nanometal.

The detailed technology and preferred embodiments of the present invention are described in detail in the following paragraphs accompanying the appended drawings for people skilled in this field to well appreciate the purposes, technical features and advantages of the claimed invention.

### BRIEF DESCRIPTION OF THE DRAWINGS

FIG. 1A shows the UV-vis-NIR absorption spectra of five embodiments of the present invention, i.e., dispersions A1 to A5 as exemplified in Example 1;

FIG. 1B shows the UV-vis-NIR absorption spectra of six embodiments of the present invention, i.e., dispersions B1 to B6 as exemplified in Example 1;

FIG. 2A shows the TEM image of dispersion A2 as exemplified in Example 1;

FIG. 2B shows the TEM image of dispersion A4 as exemplified in Example 1;

FIG. 2C shows the TEM image of dispersion A5 as exemplified in Example 1;

FIG. 2D shows the TEM image of dispersion B6 as exemplified in Example 1;

FIG. 3 shows the UV-vis-NIR absorption spectra of five embodiments of the present invention, i.e., dispersions C1 to C5 as exemplified in Example 2;

FIG. 4 shows the TEM image of dispersion C3 as exemplified in Example 2;

FIG. 5A shows the UV-vis-NIR absorption spectra of six embodiments of the present invention, i.e., dispersions D1 to D6 as exemplified in Example 3;

FIG. 5B shows the UV-vis-NIR absorption spectra of five embodiments of the present invention, i.e., dispersions E1 to E5 as exemplified in Example 3;

FIG. 5C shows the UV-vis-NIR absorption spectra of six embodiments of the present invention, i.e., dispersions F1 to F6 as exemplified in Example 3;

FIG. 5D shows the UV-vis-NIR absorption spectra of five embodiments of the present invention, i.e., dispersions G1 to G5 as exemplified in Example 3;

FIG. 6A shows the TEM image of dispersion E4 as exemplified in Example 3;

FIG. 6B shows the TEM image of dispersion E5 as exemplified in Example 3;

FIG. 6C shows the TEM image of dispersion F2 as exemplified in Example 3;

FIG. 6D shows the TEM image of dispersion F3 as exemplified in Example 3;

FIG. 6E shows the TEM image of dispersion F4 as exemplified in Example 3;

FIG. 6F shows the TEM image of dispersion F5 as exemplified in Example 3;

FIG. 7A shows the UV-vis-NIR absorption spectra of five embodiments of the present invention, i.e., dispersions H1 to H5 as exemplified in Example 4;

FIG. 7B shows the UV-vis-NIR absorption spectra of five embodiments of the present invention, i.e., dispersions I1 to I5 as exemplified in Example 4; and

FIG. 8 shows the TEM image of dispersion I5 as exemplified in Example 4.

### DESCRIPTION OF THE PREFERRED EMBODIMENT

The following will describe some embodiments of the present invention in detail.

However, without departing from the spirit of the present invention, the present invention may be embodied in various embodiments and should not be limited to the embodiments described in the specification. In addition, unless otherwise stated herein, the terms “a (an)”, “the” or the like used in this specification (especially in the Claims hereinafter) shall be understood to encompass both the singular form and the plural form.

The present invention provides a method for preparing a nanometal dispersion which has the advantages of simple preparation process, good applicability, and low cost. In addition, the method does not produce any adverse by-products. Furthermore, the nanometal thus obtained is characterized by having multimorphology, high biocompatibility, and good dispersibility for easy restoration and application.

In the method according to the present invention, a proper amount of a metal compound containing a metal ion is dissolved into a first solvent to provide a first solution, and a proper amount of inulin is dissolved into a second solvent to provide a second solution. The first solvent and the second solvent are independent polar solvents, for example ultrapure water or deionized water, and can be the same or different. Then, the first solution and the second solution are mixed to form a third solution that is subjected to a reduction-oxidation reaction via providing energy thereto.

The metal ion contained in the first solution is used as the source for synthesizing a nanometal. In general, the metal ion suitable for the present invention is a transition metal ion and/or noble metal ion such as selected from a group consisting of a gold ion, a silver ion, a palladium ion, a platinum ion, a rhodium ion, a ruthenium ion, an osmium ion, an iridium ion, a rhodium ion, a copper ion, a nickel ion, a tin ion, and combinations thereof. Optionally, one or more metal compounds, such as a transition metal-containing compound and/or a noble metal-containing compound, can be used to provide the metal ion for the method of the present invention. For instance, the metal compound can be selected from a group consisting of chloroauric acid (HAuCl₄), bromoauric acid (HAuBr₄), silver nitrate (AgNO₃), palladium chloride (PdCl₂), copper chloride (CuCl₂), copper sulfate (CuSO₄), platinum chloride (PtCl₂), tin chloride (SnCl₂), rhodium chloride (RhCl₂), nickel chloride (NiCl₂), and combinations thereof. In one embodiment of the present invention, chloroauric acid is used.

As described above, the metal ion is used for synthesizing the desired nanometal. Thus, if the concentration of the metal ion in the first solution is too low, it would be difficult to form a nanometal with the desired particle size and morphology and the yield would be low. On the contrary, if the concentration is too high, metal aggregation can easily occur and result in high costs. Therefore, in the method of the present invention, the concentration of metal ion in the first solution is generally controlled to range from about 0.05 mM to about 20 mM, and preferably from about 0.5 mM to about 15 mM.

In the method of the present invention, the inulin in the second solution is used to reduce the metal ion back to a metal, so that the metal can grow continually to the desired size and morphology. Without being bound by theory, it is believed that inulin has good water solubility and could completely unfold itself in water; therefore, it could effectively prevent the growing metal particulate and formed nanometal from aggregating, which is advantageous to nanometal synthesis, and the obtained nanometal dispersion would have good dispersibility and can be easily restored and applied. Furthermore, inulin is a polysaccharide composed of fructose units that is environment friendly, while having an excellent biocompatibility and high degradability as compared with many other reducing agents commonly used in the preparation of a nanometal such as citric acid and sodium borohydride. By using inulin, the applicability of the nanometal dispersion especially in the field of medical engineering can be greatly enhanced. For example, the nanometal prepared by the present invention can be used as a biomaterial, drug delivery vehicle, and a tool for biodetection. Moreover, as will be explained below, the preparation method using inulin according to the present invention would simplify the operation procedure to obtain a nanometal and improve its yield.

In general, in the method of the present invention, only a small amount of inulin is required to conduct a reduction-oxidation reaction. If an excessive amount of inulin is used, it would not improve the yield of the nanometal and would easily lead to solubility difficulty and high costs. Therefore, according to method of the present invention, the concentration of inulin in the second solution is generally controlled to range from about 2 mM to about 18 mM, and preferably from about 2 mM to about 14 mM.

According to the present invention, the particle size of the nanometal can be optionally adjusted via adjusting the mixing proportion of the metal ion and inulin. It is found that when the first solution and the second solution are mixed in amounts so that the molar ratio of the metal ion to inulin in the third solution ranges from about 0.0028 to about 10, preferably from about 0.005 to about 7.5, the productivity of the nanometal is better and the distribution of the particle size is more uniform.

It is further found that the morphology of the nanometal can also be controlled via adjusting the amount ratio of the metal ion to inulin, such as sphere-like (e.g., sugar apple-like, flower-like, polyhedrol-like, etc.), line-like (e.g., short line-like, thick string-like, thin string-like, etc.), reticular-like, and dumbbell-like, etc. In the case of using inulin to reduce chloroauric acid, when the molar ratio of metal ion to inulin ranges from about 0.02 to about 0.75, a sphere-like nanogold can be obtained; when the molar ratio ranges from about 0.8 to about 1.2, a short line-like shape nanogold can be obtained; when the molar ratio ranges from about 0.09 to about 0.5, a reticular-like nanogold can be obtained; and when the molar ratio ranges from about 0.01 to about 0.085, a dumbbell-like nanogold can be obtained.

The mixed third solution is then subjected to a reduction-oxidation reaction to form a nanometal. According to the method of the present invention, the step of providing energy to the third solution to carry out a reduction-oxidation can be conducted with the use of heat, light radiation or ray radiation. For example, one or more of the following manners could be adopted to carry out the reduction-oxidation reaction: a water bath, an oil bath, a heating plate, a microwave reaction, a UV light radiation, and a γ-ray radiation.

When using heat to conduct the reduction-oxidation reaction, the heating temperature generally ranges from about 20° C. to about 200° C. and preferably from about 55° C. to about 95° C. If the heating temperature is too low, the reduction-oxidation reaction cannot be conducted effectively and cannot provide a multimorphological nanometal. On the contrary, if the heating temperature is too high, the structure of inulin tends to be damaged and results in a negative impact. As for the heating time, it normally ranges from about 0.5 minutes to about 240 minutes and may be adjusted depending on the adopted heating temperature. As shown in the examples hereinafter, the morphology of the obtained nanometal can be controlled by the heating conditions.

When using light radiation, such as UV light, to conduct the reduction-oxidation reaction, a radiation power ranging from about 5 W to about 1500 W, and preferably ranging from about 10 W to about 1200 W is applied for about 0.5 minute to about 30 minutes. If the radiation power is too low, the reduction-oxidation reaction cannot be conducted effectively and cannot provide a multi-morphological nanometal. On the contrary, if the radiation power is too high, the structure of the inulin tends to be damaged. Similarly, the size and morphology of the nanometal can be controlled by regulating the radiation power and time.

It can be known from the above that the method of the present invention adds a small amount of inulin as the reducing site to synthesize a multimorphological nanometal via simple operations such as heat, light radiation or ray radiation; in addition, there are no adverse by-products. Furthermore, since inulin has excellent water solubility and high biodegradability and biocompatibility, it can not only impart an excellent dispersibility and thus easy restoration and application to the obtained nanometal dispersion but also enhance the acceptability of living organisms to the dispersion.

The present invention also provides a nanometal dispersion, comprising inulin and a nanometal, and preferably, prepared by the method described beforehand. The nanometal can be a transition metal or noble metal, for example, a metal selected from a group consisting of gold, silver, palladium, platinum, rhodium, ruthenium, osmium, iridium, rhodium, copper, nickel, tin, and combinations thereof. It is preferred that the species of the nanometal is selected from a group consisting of gold, silver and a combination thereof. The nanometal can be composed of one or more metals, wherein the latter may be a nanometal composed of gold coated with silver or composed of gold coated with a gold-silver alloy. Moreover, in the dispersion of the present invention, the molar ratio of the nanometal to inulin usually ranges from about 0.0028 to about 10, preferably from about 0.005 to about 7.5. As described above, the molar ratio of the nanometal to inulin is related to the morphology of the nanometal in the nanometal dispersion.

The nanometal in the nanometal dispersion of the present invention can be in various morphologies, including sphere-like (e.g., sugar apple-like, flower-like, polyhedrol-like, etc.), line-like (e.g., short line-like, thick string-like, thin string-like, etc.), reticular-like, and dumbbell-like, etc.

The nanometal in the dispersion of present invention has multi-morphology and thus, exhibits high applicability. In addition, since the inulin in the nanometal dispersion of the present invention can prevent the nanometal from aggregating, the dispersion has excellent dispersibility and can be easily restored.

The present invention will be further illustrated with reference to the following examples. However, these examples are only provided for illustration purposes, but not to limit the scope of the present invention. It is obvious to those skilled in the art that various changes and modifications can be made within the technical spirit of the present invention, and thus, it is apparent that these changes and modifications are included in the scope of the appended claims and their equivalents. The experiment methods and instruments are described as follows.

[UV-Vis-NIR Spectrum Analysis]

The absorption spectra of the nanometal dispersion were determined by an UV-Vis-NIR spectrophotometer (UV-1700, SHIMADZU, Japan) under the wavelength from 400 to 1000 nm.

[TEM Analysis]

The samples were put into an ultrasonic vibration machine to vibrate for 2 hours to uniformly separate the particles. Then, the appropriate amount of the samples was dropped on a silicon chip and put in an oven to be dried. The surface morphology of the samples was observed by a field emission transmission electron microscopy (JEOL JEM-2010).

### EXAMPLE

### Examples 1

### Preparation of Nanometal Dispersion

(1) Sample flasks were rinsed first with aqua regia (hydrochloric acid:nitric acid=3:1) then with ultrapure water, and then dried and ready for use.
(2) Chloroauric Acid (HAuCl₄) was dissolved in ultrapure water to form a gold ion solution with the concentration shown in Tables 1 and 2.
(3) Inulin powder was dissolved in ultrapure water and stirred at 120 rpm to form an inulin solution with a concentration listed in Tables 1 and 2.
(4) The metal ion solution prepared in step (2) was added into the inulin solution prepared in step (3) according to the amounts listed in Tables 1 and 2.
(5) The mixed solution was heated according to the amounts listed in Tables 1 and 2 to conduct a reduction-oxidation reaction with a water bath to obtain dispersions A1 to A5 and B1 to B6.

TABLE 1

| | | | Heating | |
|---|---|---|---|---|
| Nanogold | Inulin Solution | Chloroauric Acid | time | Heating |
| Dispersion | (2 mL) | Solution (1 mL) | (minutes) | Temperature |
| A1 | 10 mM | 0.1 mM | 30 | 95° C. |
| A2 | 10 mM | 0.5 mM | 30 | 95° C. |
| A3 | 10 mM | 0.7 mM | 30 | 95° C. |
| A4 | 10 mM | 0.75 mM | 30 | 95° C. |
| A5 | 10 mM | 1 mM | 30 | 95° C. |

TABLE 2

| | | | Heating | |
|---|---|---|---|---|
| Nanogold | Inulin Solution | Chloroauric Acid | time | Heating |
| Dispersion | (2 mL) | Solution (1 mL) | (minutes) | Temperature |
| B1 | 18 mM | 0.3 mM | 30 | 95° C. |
| B2 | 18 mM | 0.5 mM | 30 | 95° C. |
| B3 | 18 mM | 0.7 mM | 30 | 95° C. |
| B4 | 18 mM | 0.75 mM | 30 | 95° C. |
| B5 | 18 mM | 1 mM | 30 | 95° C. |
| B6 | 18 mM | 1.5 mM | 30 | 95° C. |

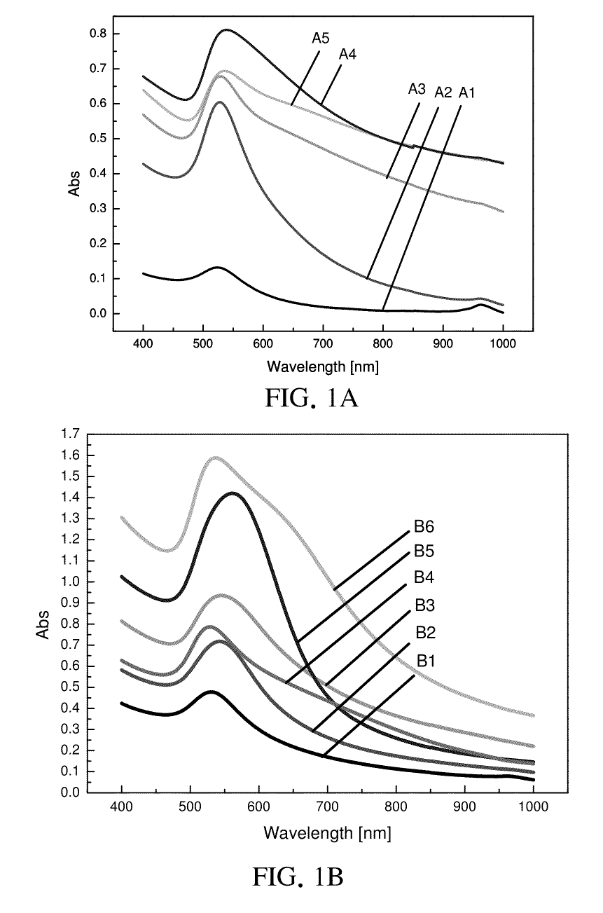

The UV-Vis-NIR absorption spectrum analysis and SEM analysis of dispersions A1 to A5 and B1 and B6 were performed according to the aforesaid methods. All UV-Vis-NIR absorption spectra of dispersions A1 to A5 in FIG. 1A and dispersions B1 to B6 in FIG. 1B show an absorption peak at 520 nm wavelength (i.e. surface plasmon resonance of gold nanoparticles), indicating that the golden ions were reduced to nanogolden particles. It also can be found that when the concentration of chloroauric acid increases, the yield of the nanogold also increases.

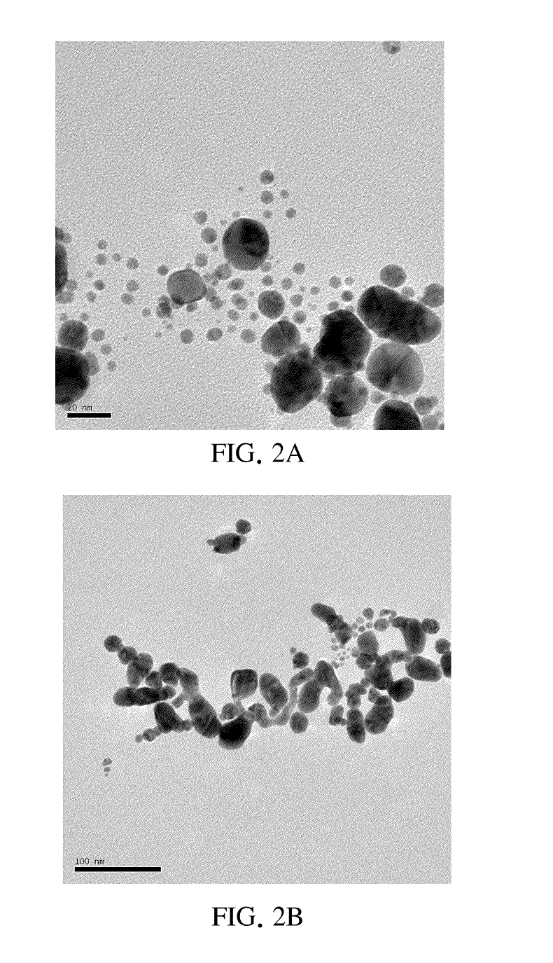

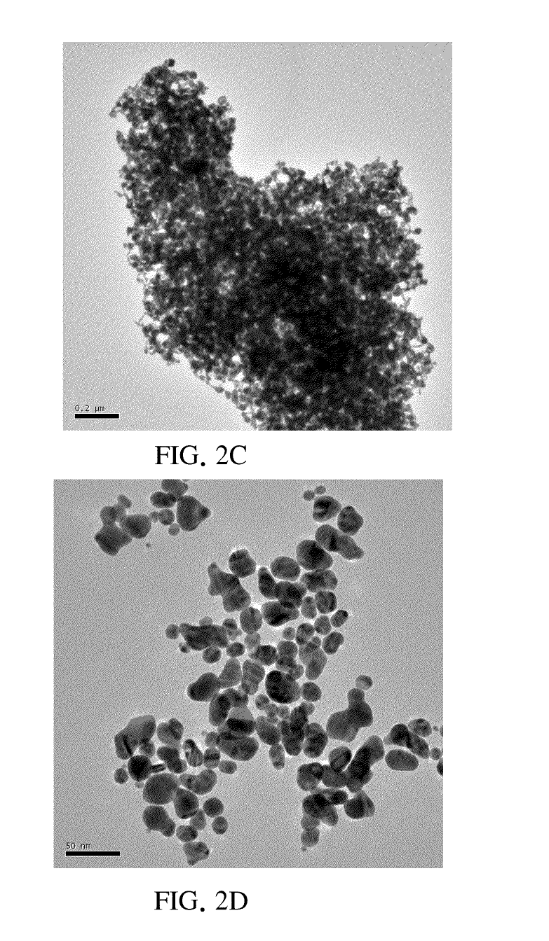

The SEM images of dispersions A2, A4 and A5 are respectively shown in FIGS. 2A to 2C, indicating that when the concentration of chloroauric acid is lower (0.5 mM), the obtained nanogold is sphere-like, and as the concentration of chloroauric acid increases, the nanogold becomes short line-like, and when the concentration of chloroauric acid is increased to 1 mM, the nanogold is formed in a reticular-like shape. The SEM image of dispersions B6 is shown in FIG. 2D, indicating that the obtained nanogold is dumbbell-like under this condition.

### Example 2

### Effects of Inulin Consumption

Dispersions C1 to C5 were prepared and analyzed using the reacting components, amounts and heating conditions listed in Table 3 and according to the preparation process and analysis process indicated above.

TABLE 3

| | | | Heating | |
|---|---|---|---|---|
| Nanogold | Inulin Solution | Chloroauric Acid | time | Heating |
| Dispersion | (2 mL) | Solution (1 mL) | (minutes) | Temperature |
| C1 | 2 mM | 0.75 mM | 30 | 95° C. |
| C2 | 6 mM | 0.75 mM | 30 | 95° C. |
| C3 | 10 mM | 0.75 mM | 30 | 95° C. |
| C4 | 14 mM | 0.75 mM | 30 | 95° C. |
| C5 | 18 mM | 0.75 mM | 30 | 95° C. |

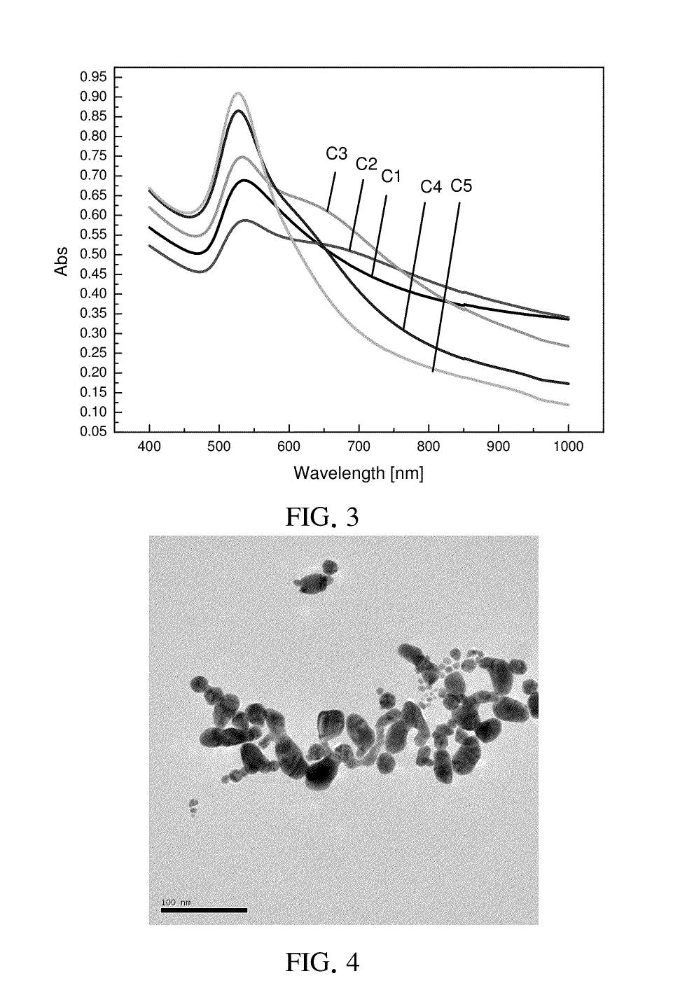

The UV-Vis-NIR absorption spectra of dispersions C1 to C5 are shown in FIG. 3, indicating that when the concentration of inulin increases, the yield of the nanogold also increases substantially. The SEM image of dispersions C3 is shown in FIG. 4, indicating that the obtained nanogold is short line-like under this condition.

### Example 3

### Effects of Heating Time

Dispersions D1 to D6, E1 to E6, F1 to F6, and G1 to G5 were prepared and analyzed using the reacting components, amounts and heating conditions listed in Tables 4 to 7 respectively and according to the preparation process and analysis process indicated above.

TABLE 4

| | | | Heating | Water Bath |
|---|---|---|---|---|
| Nanogold | Inulin Solution | Chloroauric Acid | time | Heating |
| Dispersion | (2 mL) | Solution (1 mL) | (minutes) | Temperature |
| D1 | 2 mM | 0.75 mM | 3 | 95° C. |
| D2 | 2 mM | 0.75 mM | 5 | 95° C. |
| D3 | 2 mM | 0.75 mM | 10 | 95° C. |
| D4 | 2 mM | 0.75 mM | 30 | 95° C. |
| D5 | 2 mM | 0.75 mM | 60 | 95° C. |
| D6 | 2 mM | 0.75 mM | 180 | 95° C. |

TABLE 5

| | | | Heating | Water Bath |
|---|---|---|---|---|
| Nanogold | Inulin Solution | Chloroauric Acid | time | Heating |
| Dispersion | (2 mL) | Solution (1 mL) | (minutes) | Temperature |
| E1 | 10 mM | 0.75 mM | 3 | 95° C. |
| E2 | 10 mM | 0.75 mM | 5 | 95° C. |
| E3 | 10 mM | 0.75 mM | 10 | 95° C. |
| E4 | 10 mM | 0.75 mM | 30 | 95° C. |
| E5 | 10 mM | 0.75 mM | 60 | 95° C. |
| E6 | 10 mM | 0.75 mM | 180 | 95° C. |

TABLE 6

| | | | Heating | Water Bath |
|---|---|---|---|---|
| Nanogold | Inulin Solution | Chloroauric Acid | time | Heating |
| Dispersion | (2 mL) | Solution (1 mL) | (minutes) | Temperature |
| F1 | 10 mM | 1 mM | 1 | 95° C. |
| F2 | 10 mM | 1 mM | 5 | 95° C. |
| F3 | 10 mM | 1 mM | 10 | 95° C. |
| F4 | 10 mM | 1 mM | 20 | 95° C. |
| F5 | 10 mM | 1 mM | 30 | 95° C. |
| F6 | 10 mM | 1 mM | 60 | 95° C. |

TABLE 7

| | | | Heating | Water Bath |
|---|---|---|---|---|
| Nanogold | Inulin Solution | Chloroauric Acid | time | Heating |
| Dispersion | (2 mL) | Solution (1 mL) | (minutes) | Temperature |
| G1 | 18 mM | 0.75 mM | 3 | 95° C. |
| G2 | 18 mM | 0.75 mM | 5 | 95° C. |
| G3 | 18 mM | 0.75 mM | 10 | 95° C. |
| G4 | 18 mM | 0.75 mM | 30 | 95° C. |
| G5 | 18 mM | 0.75 mM | 60 | 95° C. |

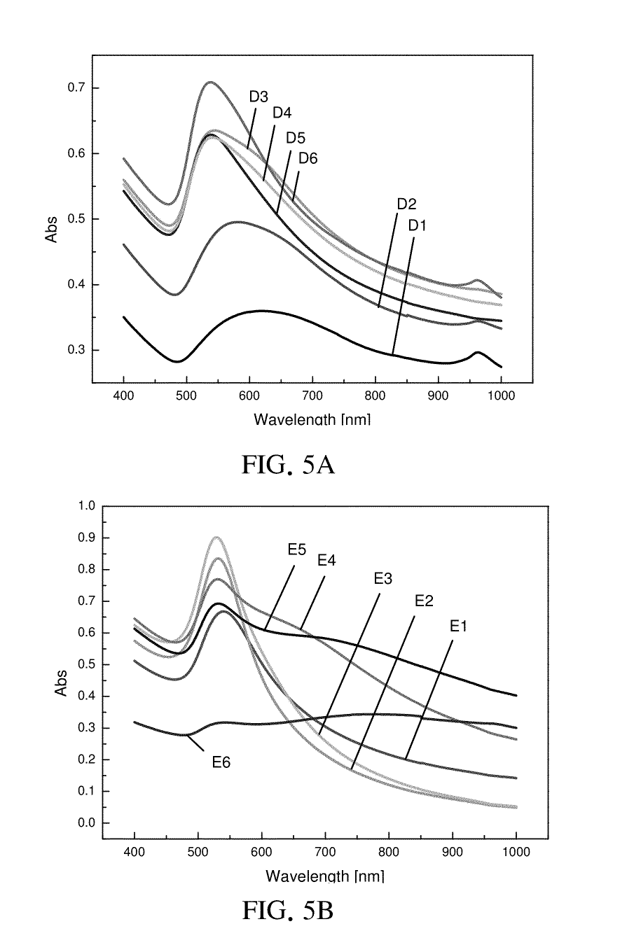

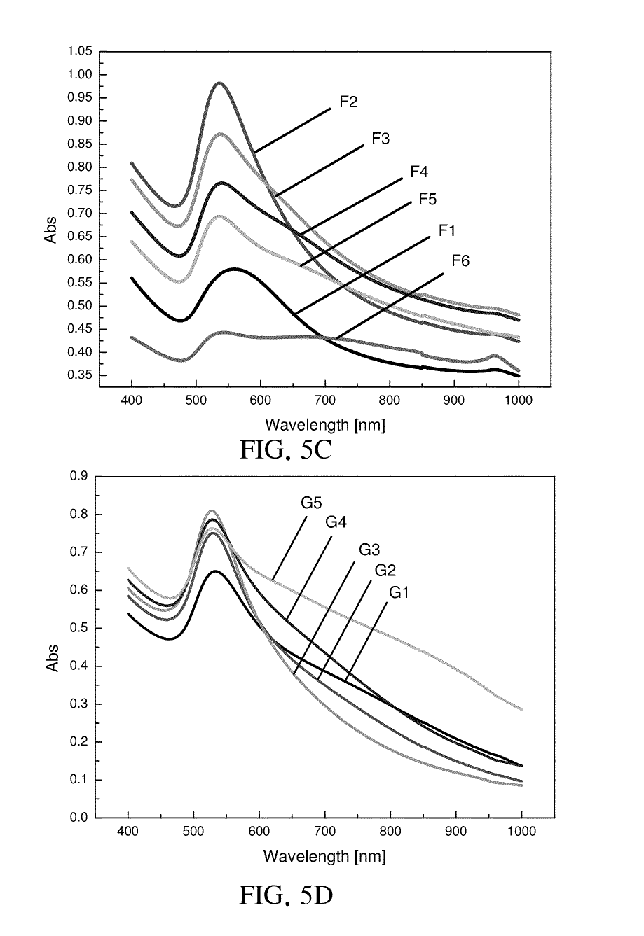

The UV-Vis-NIR absorption spectra of dispersions D1 to D6 are shown in FIG. 5A; the UV-Vis-NIR absorption spectra of dispersions E1 to E6 are shown in FIG. 5B; the UV-Vis-NIR absorption spectra of dispersions F1 to F6 are shown in FIG. 5C; and the UV-Vis-NIR absorption spectra of dispersions G1 to G5 are shown in FIG. 5D. It can be found that the yield of the nanogold is varied with the heating time. For example, when the concentration of inulin solution is 2 mM and the concentration of chloroauric acid solution is 0.75 mM (dispersions D1 to D6), the yield of the nanogold is highest when the heating time is 180 minutes at 95° C.; when the concentration of inulin solution is 10 mM and the concentration of chloroauric acid solution is 0.75 mM (dispersions E1 to E6), the yield of the nanogold is highest when the heating time is 10 minutes at 95° C.; and when the concentration of inulin solution is 10 mM and the concentration of chloroauric acid solution 1 mM (dispersions F1 to F6), the yield of the nanogold is highest when the heating time is 5 minutes at 95° C.; and when the concentration of inulin solution is 18 mM and the concentration of chloroauric acid solution is 0.75 mM (dispersions G1 to G6), the yield of the nanogold is highest when the heating time is 10 minutes at 95° C.

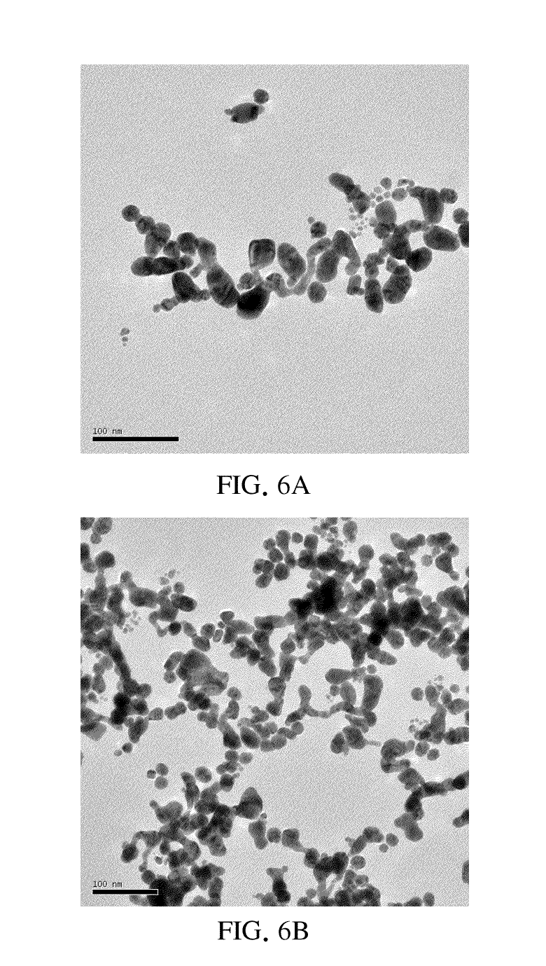

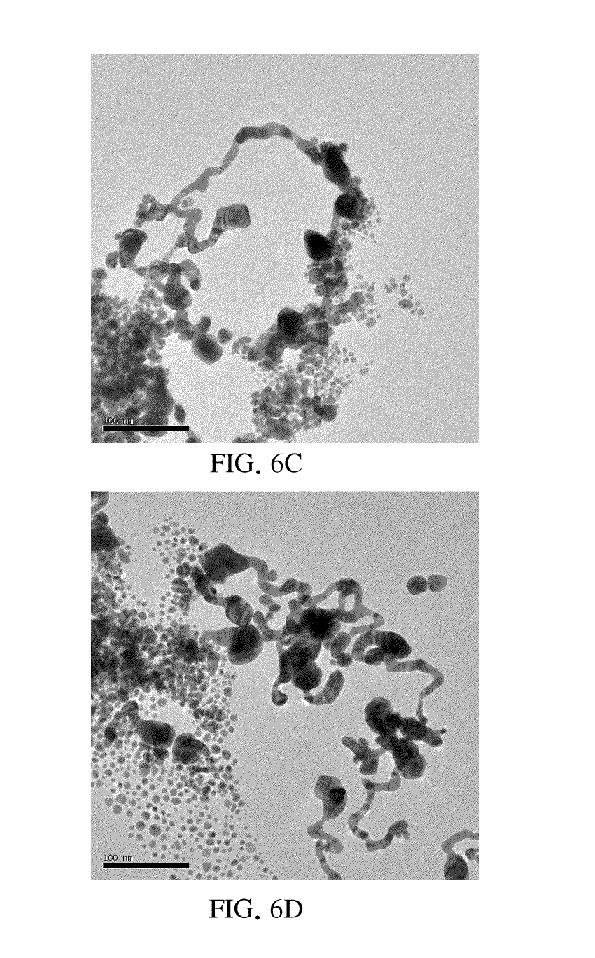

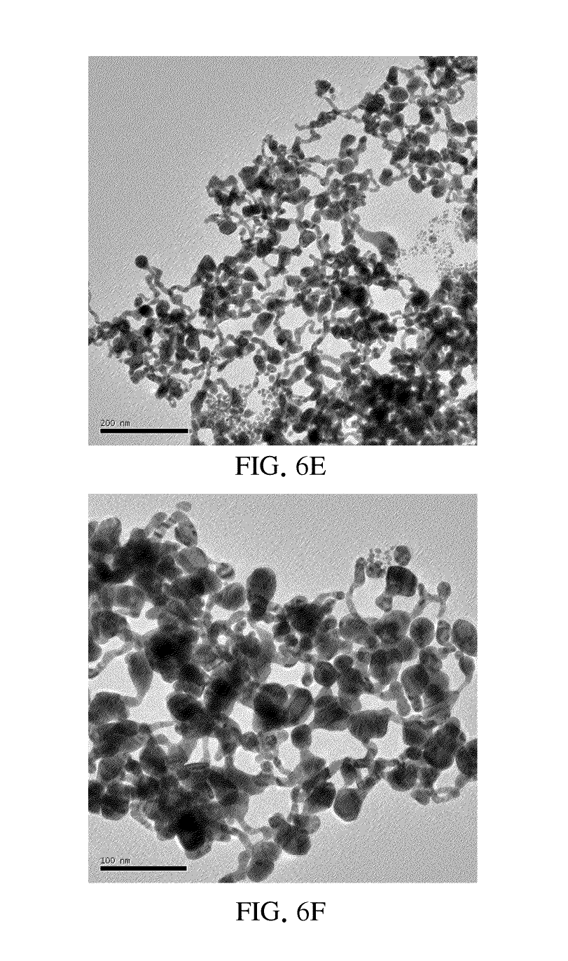

In FIGS. 6A to 6F, the TEM images of dispersions E4, E5, and F2 to F5 show the multi-morphology of the obtained nanogold. The obtained nanogold shows a short line-like shape when the concentration of inulin solution is 10 mM, the concentration of chloroauric acid solution is 0.75 mM, and the heating time is about 30 minutes or 60 minutes at 95° C. (FIGS. 6A and 6B, dispersion E4 and E5). The obtained nanogold shows a line-like shape when the concentration of inulin solution is 10 mM, the concentration of chloroauric acid solution is 1 mM, and the heating time is about 5 minutes, 10 minutes or 30 minutes at 95° C. (FIGS. 6C, 6D and 6F, dispersion F2, F3 and F5). In addition, when the concentration of inulin solution is 10 mM, the concentration of chloroauric acid solution is 1 mM, and the heating time is about 20 minutes, the obtained nanogold shows a reticular-like shape at 95° C. (FIG. 6E, dispersion F4).

### Example 4

### Effects of Heating Temperature

Dispersions H1 to H5 and I1 to I5 were prepared and analyzed using the reacting components, amounts and heating conditions listed in Tables 8 and 9 respectively and according to the preparation process and analysis process indicated above.

TABLE 8

| | | | Heating | |
|---|---|---|---|---|
| Nanogold | Inulin Solution | Chloroauric Acid | time | Heating |
| Dispersion | (2 mL) | Solution (1 mL) | (minutes) | Temperature |
| H1 | 2 mM | 0.75 mM | 30 | 55° C. |
| H2 | 2 mM | 0.75 mM | 30 | 65° C. |
| H3 | 2 mM | 0.75 mM | 30 | 75° C. |
| H4 | 2 mM | 0.75 mM | 30 | 85° C. |
| H5 | 2 mM | 0.75 mM | 30 | 95° C. |

TABLE 9

| | | | Heating | |
|---|---|---|---|---|
| Nanogold | Inulin Solution | Chloroauric Acid | time | Heating |
| Dispersion | (2 mL) | Solution (1 mL) | (minutes) | Temperature |
| I1 | 10 mM | 0.75 mM | 30 | 55° C. |
| I2 | 10 mM | 0.75 mM | 30 | 65° C. |
| I3 | 10 mM | 0.75 mM | 30 | 75° C. |
| I4 | 10 mM | 0.75 mM | 30 | 85° C. |
| I5 | 10 mM | 0.75 mM | 30 | 95° C. |

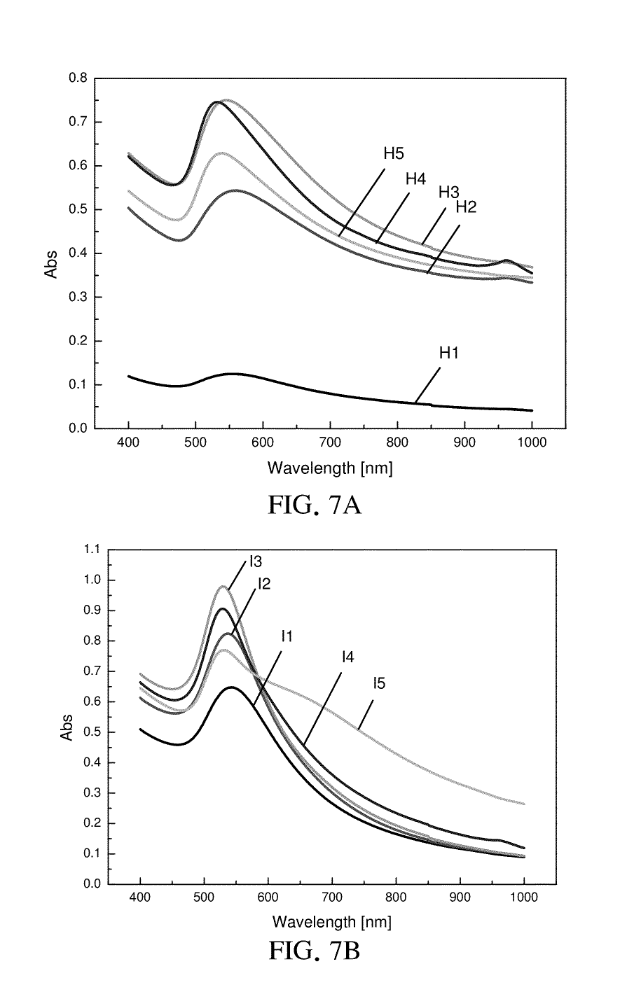

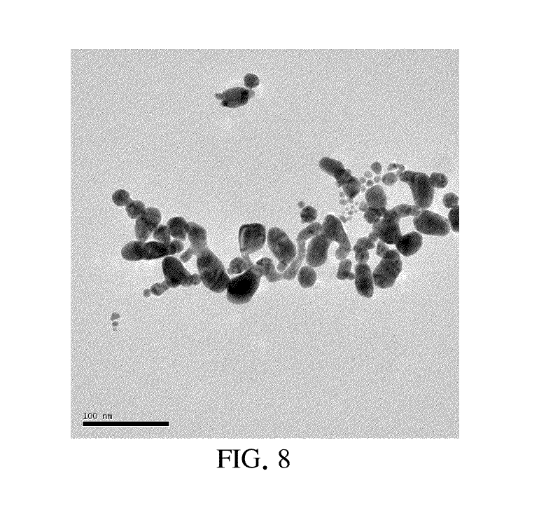

The UV-Vis-NIR absorption spectra of dispersions H1 to H5 are shown in FIG. 7A and the UV-Vis-NIR absorption spectra of dispersions I1 to I5 are shown in FIG. 7B. It can be found that when the heating time increased from 55° C. to 75° C., the yield of the nanogold also increases. In FIG. 8, the TEM image of dispersions I5 shows a short-line shape when the heating time is about 30 minutes at 95° C.

The above results indicate that the morphology of the nanometals prepared by the method of the present invention can be varied according to the ratio between the molar ratio of the metal ion to inulin, heating time, or heating temperature.

What is claimed is:

1. A method for preparing a nanometal dispersion, comprising:
providing a first solution containing a metal ion;
providing a second solution containing inulin;
mixing the first solution and the second solution to provide a third solution; and
providing energy to the third solution to conduct a reduction-oxidation reaction to form a nanometal therein.

2. The method as claimed in claim 1, wherein the metal ion is a transition metal ion.

3. The method as claimed in claim 2, wherein the metal ion is selected from a group consisting of a gold ion, a silver ion, a palladium ion, a platinum ion, a rhodium ion, a ruthenium ion, an osmium ion, an iridium ion, an rhodium ion, a copper ion, a nickel ion, a tin ion, and combinations thereof.

4. The method as claimed in claim 3, wherein the metal ion is a gold ion, a silver ion or a combination thereof.

5. The method as claimed in claim 1, wherein the step of providing the first solution comprises dissolving a metal compound containing the metal ion into a first solvent, and the step of providing the second solution comprises dissolving inulin into a second solvent, wherein the first solvent and the second solvent are independently a polar solvent.

6. The method as claimed in claim 5, wherein the first solvent and the second solvent are independently selected from a group consisting of ultra-pure water and de-ionized water.

7. The method as claimed in claim 1, wherein the first solution and the second solution are mixed in such an amount that the molar ratio of the metal ion to inulin is from about 0.0028 to about 10.

8. The method as claimed in claim 7, wherein the molar ratio of the metal ion to inulin is from about 0.005 to about 7.5.

9. The method as claimed in claim 1, wherein the energy is provided by at least one of the following means: a water bath, an oil bath, a heating plate, a microwave reaction, an UV light radiation, and a γ-ray radiation.

10. The method as claimed in claim 1, wherein the step of providing energy to the third solution to conduct reduction-oxidation reaction comprises heating the third solution to a temperature ranging from about 20° C. to about 200° C. and maintaining at the temperature for about 0.5 minutes to about 240 minutes.

11. The method as claimed in claim 10, wherein the third solution is maintained at about 55° C. to about 95° C.

12. A nanometal dispersion comprising inulin and a nanometal.

13. The nanometal dispersion as claimed in claim 12, wherein the nanometal is transition metal.

14. The nanometal dispersion as claimed in claim 13, wherein the nanometal is selected from a group consisting of gold, silver, palladium, platinum, rhodium, ruthenium, osmium, iridium, rhodium, copper, nickel, tin and combinations thereof.

15. The nanometal dispersion as claimed in claim 14, wherein the metal is gold, silver or a combination thereof.

16. The nanometal dispersion as claimed in claim 12, wherein the molar ratio of the nanometal to inulin is from about 0.0028 to about 10.

17. The nanometal dispersion as claimed in claim 16, wherein the molar ratio of the nanometal to inulinis from about 0.005 to about 7.5.

18. The nanometal dispersion as claimed in claim 12, which is prepared by a method comprising providing a first solution containing a metal ion; providing a second solution containing inulin; mixing the first solution and the second solution to provide a third solution; and providing energy to the third solution to conduct reduction-oxidation reaction to form a nanometal therein.

19. The nanometal dispersion as claimed in claim 12, wherein the nanometal is in a shape selected from a group consisting of sphere shape, short-line shape, reticular shape, and dumbbell shape.
